(19) United States
(12) Patent Application Publication
(10) Pub. No.: US 2005/0075727 A1
(43) Pub. Date: Apr. 7, 2005

(54) Mitral valve prosthesis

(76) Inventor: David John Wheatley, Glasgow (GB)

Correspondence Address: NIXON & VANDERHYE, PC, 1100 N GLEBE ROAD, 8TH FLOOR, ARLINGTON, VA, 22201-4714, US

(21) Appl. No.: 10/493,950

(22) PCT Filed: Oct. 29, 2002
(86) PCT No.: PCT/GB02/04873

(30) Foreign Application Priority Data
Oct. 29, 2001 (GB) 0125925.8

(51) Int. Cl. A61F 2/24

(52) U.S. Cl. 623/2.17; 623/902

(57) ABSTRACT

The present invention relates to a mitral valve prosthesis comprising flexible leaflet-like elements with curved coapting surfaces and means for maintaining continuity of the valve when inserted into the mitral annulus, which mimics the continuity between the papillary muscles, the chordae tendineae, the mitral valve leaflets and the mitral annulus of a natural valve. The present invention also relates to a method of fitting such a prosthesis to heart of a patient.

### FIELD OF THE INVENTION

This invention relates to a mitral valve prosthesis, and to a method of fitting such a prosthesis to the heart of a patient.

### BACKGROUND OF THE INVENTION

The mitral valve controls blood flow at the orifice between the left atrium (a receiving chamber for blood coming from the lungs) and the left ventricle (the high pressure muscular pumping chamber of the heart), which ejects blood into the aorta (the main artery which distributes blood around the body through its branches). When the left ventricular muscle relaxes, the cavity of the left ventricle expands to receive blood through the open mitral valve (FIG. 1); when the left ventricle 4 muscle contracts, the mitral valve leaflets 6, 7 close and the blood in the left ventricle 4 is expelled through the open aortic valve 15 into the aorta 14 (FIG. 2). The mitral valve has the function of preventing reverse blood flow back into the left atrium 8 when the left ventricle is contracting.

The mitral valve has two major leaflets—the anterior and the posterior leaflets 6, 7 (small leaflets may variably be present between these two). The leaflets are flexible and are attached to the annulus (a tough, fibrous region surrounding the orifice of the mitral valve). Arising from the leaflets are fibrous strings (chordae tendineae). The most important of these (the primary chordae tendineae 10) run from the free edges of the leaflets 6, 7 to two specialised thickened areas of the left ventricular muscle known as the antero-lateral and postero-medial papillary muscles 12. The medial half of both anterior and posterior leaflets attach to the postero-medial papillary muscle, and the lateral halves of the anterior and posterior leaflets are attached to the antero-lateral papillary muscle. The papillary muscles 12 may have multiple heads, though generally a main muscle mass is distinguishable for each muscle.

The mitral valve may be abnormal as a result of congenital deformity (rarely) or as a result of rheumatic fever or degenerative change. When the valve becomes scarred, thickened or calcified (as is common in rheumatic valves) the valve becomes stenotic (functionally narrowed) and causes obstruction to blood flow, with the result that the lungs become congested and the patient breathless. When the valve becomes dilated or when chordae rupture the valve leaks, causing increased pressure in the left-atrium and lung congestion, as well as a fall in the efficiency of the heart.

Many abnormal mitral valves can be repaired at surgery, but a large number are so abnormal as to preclude repair. Replacement devices (prostheses) are of two general categories: the biological valves are of animal origin and mimic the natural aortic valve, being either the aortic valve of a pig or a similar device made from pericardium of a calf (both devices mounted on frames and suitably treated to ensure sterility and reasonable durability); mechanical valves are made of metal alloys or carbon, and do not resemble the natural valves. The mechanical valves are commonly of the tilting disc or bileaflet variety (two hemidiscs) where the occluding disc or hemidiscs are restrained or hinged within a circular housing, which is itself surrounded by a fabric sewing ring to facilitate sewing into the mitral orifice once the diseased valve has been removed.

Current prosthetic valve designs available for clinical implantation are, generally, not ideal. In addition to the general problems associated with heart valve prostheses (biological versions are prone to degenerate and calcify and have limited durability in the young in particular; mechanical versions require life-long anticoagulation to minimise the risk of their causing clotting of the blood, thromboembolism), when they are used in the mitral position, additional problems arise. In the aortic position, flow through both types of prosthesis reasonably approximates natural flow (particularly for bioprostheses, which mimic the natural aortic valve in their construction). In the mitral position, flow is more complex, involving vortices in the left ventricular cavity that are not replicated by current prostheses. This may well contribute to the added thrombogenicity of mitral prostheses in general. Clinical prosthetic valves are dissimilar to the natural mitral valve with the consequence of loss of chordal function, which affects left ventricular function deleteriously over time. Problems related to anticoagulation therapy required by mechanical valves are magnified in the mitral position compared with aortic valve replacement. Bioprostheses project into the ventricle with the potential for causing direct damage to the ventricular muscle. Developments in Cardiology promise the elimination of atrial fibrillation, a common complication of mitral valve disease requiring anticoagulation. This would make a non-thrombogenic mitral valve even more widely applicable.

A number of potential mitral valve prostheses have been described. The earliest published description of a prosthesis mimicking the natural mitral valve was in 1960 (Braunwald NS, Cooper T, Morrow AG. Experimental replacement of the mitral valve with a flexible polyurethane foam prosthesis. Trans Am Soc Artif Intern Organs 1960; 6:312-322.). The valve leaflets were fabricated from an open cell polyurethane foam reinforced with a fine knit Dacron fabric. After trimming of the leaflet shapes, chordae made of woven Teflon fabric tape were stitched to the leaflets. During experimental implant studies in dogs, the chordae were attached through the ventricular wall near the papillary muscle insertions and manually adjusted while the dogs were still on partial perfusion and then fixed to the ventricular wall muscle using Teflon felt pieces. Clinical application of this valve has been described in five patients (Braunwald NS, Cooper T, Morrow AG. Clinical and Experimental Replacement of the Mitral Valve. In: Merendino K A (Ed.), “Prosthetic Valves for Cardiac Surgery”, Charles C Thomas, Springfield, Ill., 1961, pp 307-399.). Three patients died of operative complications. One further patient survived for 14 h, developing progressive refractory hypotension. No specific cause of death was identified. The fifth patient survived for approximately three months after operation. Again, no specific cause of death was identified, and no valve-associated pathology noted. Complications associated with the method of tensioning the chordae of this design were noted, including uncontrollable bleeding from the ventricle in one patient.

International Patent WO 149217A (U.S. Pat. No. 5,910,169), Total mitral heterologous bioprosthesis to be used in mitral tricuspid heart replacement, Peredo MOV, describes a natural mitral valve harvested from animal sources (heterograft or xenograft), with the addition of pericardial tissue to support the valve annulus. The valve is harvested with the chordae and sections of papillary muscle included. The excised papillary muscle is reinforced with a patch. Unfixed homograft valves are also described. The valve dimensions are size-matched to the recipient.

U.S. Pat. No. 5,415,667, Mitral Heart Valve Replacements, Frater R W, describes an entirely flexible non-elastic bioincorporable unstented replacement mitral valve with no rigid parts projecting into the ventricle cavity. The valve is described with a D-shaped annulus, although they use a trapezium by preference to avoid bending/folding or creasing of the leaflets structures during opening and closing. The valve has four cusps: one anterior; one posterior and two lateral, with chordae extending from the leaflet edges to the papillary muscles. Either separate chordae are attached by sewing to the cusp edges or, preferably, formed integral with the leaflet cusps with integral papillary muscle attachment pieces. The cusps, chordae and papillary muscle attachment pieces are shown to be reinforced by non-elastic material running from the cusps to the attachment pieces: similar reinforcement are shown to run along the free edge or middle of the cusps. The valve material used is pericardium, or a non-biological material: reinforcement is described as using extruded polytetrafluoroethylene (ePTFE) sutures to guard against stretching. In either case, the valve material is described as allowing overgrowth of the material with fibrous tissue and endothelium to leave the prosthesis completely covered by natural host tissue. The aggregate surface area of the cusps exceeds an area circumscribed by the sewing ring so that, when the valve opens, the papillary muscles pull the chordae and the cusps deflect away from each other. The valve is a four-leaflet valve with chordae attached to or extending from the edges of the leaflet cusps, with the possibility of superficial reinforcement of the leaflet material.

U.S. Pat. No. 5,344,442, Cardiac Valve, Deac R, describes a mitral valve prosthesis formed by a tapering tube shape without separate leaflets. The free end portion is oval with the possibility of multiple integral flaps of unequal size. The tube is constructed of pericardial membranes or other suitable biological or synthetic fabric stitched together. The tubular shape extends from the annulus down to the papillary muscles to which it is attached directly to the chordal remnants at two of the “integral flaps”. The valve, while it fits the mitral position, is not a mimic of the natural mitral valve shape and does not incorporate chordal function. The attachment to the papillary muscle is made by stitching the suture lengths remaining from the stitched seams of the tubular valve, with the tissue protected by standard Teflon pledgets.

U.S. Pat. No. 4,275,469, Prosthetic Heart Valve, Gabbay S, describes a mitral valve of a more or less tubular form of essentially uniform cross-section, similar to the Deac patent described above, but with an extended single flap to form the valve opening. The tube has the option of a supporting strut to prevent leaflet prolapse. The valve material is biological in origin and the design does not mimic the natural valve. There are no chordae. The tube is directly attached to the papillary muscles, by conventional suturing techniques.

German Patent No. DE 4234127 A1, Heart Valve Prosthesis, Reichart B & Vetter H, describes a distributor mitral valve, which can be fixed to the heart wall with the aid of plastic patches. The mitral valve described is derived from a natural mitral valve from human or animal source. A plastic patch (preferably PTFE) is put over the papillary muscle heads (remnants excised with the natural valve) as an implantation aid. The natural chordae are intact and the fixation is not restricted to the locus of the papillary muscle in the recipient heart. There is an additional possibility of reinforcement of the bioprosthetic tissue by threading a plastic thread through the leaflet, using a running stitch, from the sewing ring down to the plastic patch at the fixation point. The valve described here is, essentially, a transplanted natural valve with some additions to preserve and support the tissue.

It is amongst the objects of the present invention to provide an improved heart valve prosthesis.

### SUMMARY OF THE INVENTION

According to a first aspect of the present invention, there is provided a heart valve prosthesis comprising:
a support adapted to be coupled to the mitral annulus;
a valve element coupled to the support and moveable between an open position and a closed position;
at least one continuity member having one end coupled to said support and the other end for coupling to papillary muscle; and
restraining means for securing the continuity member to an area of heart muscle and for permitting adjustment of the continuity member externally of the heart.

A significant advantage of the present invention, in use, lies in the maintenance of continuity between the papillary muscles and the mitral annulus by the provision of the at least one continuity member, such that left ventricular function is maintained. Furthermore, continuity may be optimised according to the dimensions of each individual heart due to the adjustability of the continuity member. Loss of such continuity is inherent in the process of removal of the diseased mitral valve and subsequent replacement using a conventional prosthetic heart valve.

According to a second aspect of the present invention there is provided a heart valve prosthesis comprising:
a support adapted to be coupled to the mitral annulus;
first and second valve elements coupled to the support and moveable between an open position and a closed position, the elements defining curved coapting surfaces, each surface having at least two points of inflection; and
at least one continuity member having one end coupled to said support and the other end adapted for coupling to papillary muscle.

This second aspect of the present invention provides a form which allows flow of blood through the open valve prosthesis, the form of the valve elements providing an open flow area comparable to the open flow area of the natural valve, despite the use of relatively stiff synthetic material to form the valve elements.

Preferably, the support has a ring-like or annular structure. The support may comprise a semi-rigid structure, and may comprise a suitable metal.

Conveniently, the support is incorporated into a fabric sewing ring. Preferably, said fabric sewing ring is formed of polytetrafluoroethylene or the like. Such a sewing ring facilitates sewing of the mitral valve prosthesis into the mitral orifice of the heart once the diseased or defective valve has been removed.

Preferably, a plurality of valve elements is provided. The valve elements may be flexible and most preferably mimic the anterior and posterior leaflets of a working biological valve in vivo. Most preferably, the prosthesis comprises two flexible valve elements, in the form of an anterior and a posterior leaflet. Preferably, the valve elements comprise elongate reinforcing elements, which may take the form of mono- or multifilament (twisted or braided) strands or fibres, most preferably of a strong but flexible biocompatible material, such as polytetrafluoroethylene (PTFE), polypropylene or carbon fibre. Typically, the strands are attached around the support and radiate therefrom. These primary reinforcing strands may be mutually coupled by secondary strands, which preferably encircle the primary strands to create a net-like structure. Preferably, this net is embedded in or otherwise coupled to a flexible material, which forms the surfaces of the valve elements. The material may be selected for flexibility and mechanical properties compatible with the reinforcing strands. Most preferably, the net is embedded within a biostable polymer, for example polyurethane, polypropylene or a suitable biopolymer. Conveniently, the net created is a size and shape corresponding to the valve leaflets as found in vivo.

Alternatively, short strand discontinuous fibres of high aspect ratio may be used to provide the secondary reinforcement of the leaflet, using materials of suitable composition, for example polysulphone, polyurethane, or carbon nano-tubes. Conveniently, the leaflet may be reinforced using a non-woven fabric of suitable material composition, for example, carbon fibre or the materials previously mentioned.

Preferably, primary reinforcing strands may be embedded in a single plane within the flexible leaflet material or may be organised in two or more separate planes with flexible polymer material between, thus providing protection for the fibres against long-term damage due to abrasion between adjacent fibres.

Preferably, the primary reinforcing strands continue beyond the valve elements and have ends adapted for coupling to papillary muscle; thus, the strands serve as said continuity members. This preferred form of the invention thus mimics the natural continuity between the papillary muscles, the chordae tendineae, the mitral valve leaflets and the mitral annulus. Moreover, this preferred form of the present invention maintains near normal blood flow across the valve and within the left ventricle, resulting in a reduction in risk of thromboembolism and thus a reduction in the thromboembolic risk associated with conventional prosthetic valves.

The heart valve prosthesis may further comprise a tubular member for receiving the other end portion of the continuity member for coupling to the papillary muscle. Preferably, the tubular member is a rigid or semi-rigid structure manufactured from a suitable polymeric material or, for example, a metal alloy such as Titanium or a shape-memory alloy such as Nitinol. Typically, the leading end of the tube is in the form of a trocar, adapted to be pushed through the heart muscle, typically the thickest portion of the papillary muscle, to the outside of the heart. Thus, the tube will typically emerge on the lower, external surface of the left ventricle of the heart. Preferably, the tube has an open mesh structure to allow tissue ingrowth and healing to occur, reinforcing the ventricular muscle tissue at the chordal attachment position. More preferably, the valve may be coated with a material to improve blood compatibility, for example, a hydrogel or fluoropolymer plasma.

Preferably, the valve elements according to the present invention have a geometry that mimics the function of anterior and posterior leaflets of a working biological valve in vivo and which is optimised to permit efficient flow of blood through the valve in an open position, whilst maintaining a seal, without leakage, when the valve is in a closed position. More preferably, the free edges of the valve elements comprise one or more folds. Advantageously, the valve elements may further comprise cut-out regions at the free edges of one or both valve elements. Conveniently, the cut-out regions may be incorporated to relieve stress at the free edge and minimise valve wear due to abrasion of the each valve element against each other over the coaptation area. Preferably, the cut-outs may be applied to one leaflet or to alternate folds in both leaflets.

Preferably, the design of the free edge of the valve element is constructed using a 3D spline curve. Conveniently, a polynomial equation of the 6^{th} order is used to define one half of the free edge curve, which is symmetrical about a centre plane as it is projected onto standard Cartesian planes in 2D form. For example, the Cartesian planes are the x-y plane, the x-z plane and y-z plane. The centre plane is the y-z plane. Generally, the 6^{th} order equation used to define the free edge curve for any embodiment, 1-fold, 2-fold, 3-fold, for example, is as follows:
f(t)=at⁶+bt⁵+ct⁴+dt³+et²+ft+g
where t is the x-, y-, or z-coordinate of a point along the free edge curve, f(t) is a function of this point, and a-g are coefficients.

The prosthesis may further comprise restraining means for securing the continuity member to an area of heart muscle of choice. Preferably, the restraining means comprises a compressible crimping clip, which may itself be restrained by a washer. Typically, the compressible crimping clip and washer are of a polymer or similar material. In other embodiments, other forms of restraining means may be provided including other mechanical fixing arrangements or non-mechanical arrangements.

Optionally, the restraining means may be integral to the tubular member. Alternatively, the restraining means may comprise a washer that allows the tubular member to be positioned from inside the ventricle and a separate washer, which is added later to position the tubular member from the outside of the ventricle.

In a third aspect of the present invention there is provided a method of inserting a heart valve prosthesis comprising a support adapted to be coupled to the mitral annulus, a valve element coupled to the support and moveable between an open position and a closed position, and at least one continuity member having one end coupled to said support and the other end adapted for coupling to papillary muscle, wherein said continuity member continues beyond the valve elements and comprises strands, which mimic the chordae tendinae of a natural valve and which are individually adjustable said method comprising the steps of:
(i) securing the support to the mitral orifice of the heart;
(ii) passing the continuity member through the papillary muscle of the heart; and
(iii) securing the continuity member to said papillary muscle.

The term “heart” according to the present invention means a mammalian heart, particularly a human heart.

In a yet further aspect of the present invention there is provided a method of inserting a heart valve prosthesis, comprising a support adapted to be coupled to the mitral annulus, a valve element coupled to the support and moveable between an open position and a closed position, and at least one continuity member having one end coupled to said support and the other end adapted for coupling to papillary muscle, wherein said continuity member continues beyond the valve elements and comprises strands, which mimic the chordae tendinae of a natural valve and which are individually adjustable said method comprising the steps of:
(a) excising a diseased valve from a patient, wherein papillary muscle tips and a rim of leaflet tissue at the mitral orifice are left intact;
(b) measuring the size of the patient's valve cavity;
(c) determining the appropriate sites within the patient's papillary muscle to receive a continuity member of the heart valve prosthesis;
(d) perforating the patient's heart at the determined papillary muscle site;
(e) securing the support to the mitral orifice of the heart;
(f) passing the continuity member through the papillary muscle of the heart; and
(g) securing the continuity member to said papillary muscle.

A tubular member may be pushed through or otherwise located in the papillary muscle to receive the continuity member. Preferably, a long needle or other suitable medical device is used to perforate the heart at the intended site, as near to the papillary muscle tips as possible, which is to receive the continuity member. Generally, a flexible guide-wire is passed through the perforated site and may be used to guide the passage of dilators, as necessary, and finally the tubular members comprising the continuity members through the site. Finally, the heart valve prosthesis may be secured in the appropriate orientation, to mimic the natural valve. Preferably, securement of the support is by sewing the support into the mitral orifice of the heart. Advantageously, this is achieved by sewing a fabric sewing ring surrounding said support into the mitral orifice of the heart. More preferably, the support means is sewn into the mitral annulus. Suturing is achieved using techniques known in the art and/or according to surgical preference and practice.

Preferably, the continuity member is adjusted while in place in the heart. The term “adjustment” according to the present invention is understood to mean slackening or tensioning. Accurate adjustment of the continuity member may be achieved by making an initial adjustment on inspection of the valve element with the heart still open. Adjustment of the continuity member may then be fine tuned with the heart closed and beating normally, before closing the chest. Preferably, the functioning of the heart valve prosthesis is visualised using echocardiography, or some other method known to those of skill in the art. The tension of the continuity member may be maintained by crimping a compressible crimping clip or other retainer on the continuity member, the clip being located on the outside of the papillary muscle. Preferably, the crimping clip is located adjacent to a washer or button also provided on the outside of the papillary muscle. The natural tension on the washer tends to prevent bleeding.

As described above, tensioning of the continuity member may be achieved under echocardiographic control to the specifics of a recipients' heart. Advantageously, this permits essentially normal coaptation of the preferred pair of valve elements, for example, in the plane of the valve annulus. In other words, the valve elements are prevented from prolapsing into the left atrium of the heart, yet are ensured of closing with sufficient mutual contact area. More preferably, the continuity member is adjusted by slackening, to the extent where it is not too loose to cause prolapse of the valve elements into the atrium of the heart. Conversely, said continuity member is tensioned avoiding overtensioning, which may interfere with valve element apposition.

Preferably, a plurality of continuity members are provided, and most preferably individual continuity members, or individual elements of continuity members, are identifiable, for example by colour coding; this facilitates selective tensioning of particular portions of the valve elements or leaflets to achieve optimal leaflet geometry.

In a further embodiment of the present invention the plurality of continuity members, which pass through the tubular member of the present invention spanning the heart muscle wall may be further passed through access tubes, which traverse the body wall. The access tube may be provided in combination with chest drainage tubes, as will normally be fitted to a patient following open heart surgery according to surgical techniques well known in the art. Advantageously, this allows the continuity member, which may be individually identifiable, to be adjusted appropriately from outside the operative field to achieve the appropriate tension. The continuity member(s) may be temporarily clamped until final adjustments are made to the tension of the continuity member(s). The temporary clamping may be achieved, for example, by a noose-like strand, which surrounds the continuity member that runs through the access tubes. Preferably, the noose-like strand is located close to the point of egress from the heart. Preferably, the noose-like strand may be tightened or loosened by tensioning or releasing the ends of the noose-like strand, which pass outside the body through a channel within the same access tubes as the continuity members.

Generally, adjustment of the continuity member(s) is undertaken manually by the surgeon. Optionally, adjustment of the continuity member(s) is aided by the use of echocardiographic imaging according to known techniques, for example, echocardiographic imaging, transoesophageal echocardiographic imaging or other forms of imaging. Adjustment of the continuity member(s), or individual strands or chordae of said continuity member(s), may be made in the operating room before closure of the chest. Advantageously, the continuity members may be adjusted after closure of the chest in the first few postoperative days by adjusting the continuity members on the outside of the body, which have been passed through tubes which transverse the body wall. This may avoid the need to reopen the chest wall and adjust the continuity members directly at the site that they span the heart wall.

Final securing of the continuity member(s) is completed once appropriate adjustment outside of the body is achieved. Securement may be actuated from outside of the body using any suitable device or mechanism. For example, the buttons on the external surface of the heart, through which the continuity member pass, may incorporate a strong, sprung C-shaped clip that is held open by a rigid segment located between the ends of the clip. Preferably, this segment may be withdrawn through the access tube once final adjustment has been confirmed, with the result that the C-shaped clip becomes O-shaped and fixes the position of the continuity member or the individual strands thereof. Once securement of the continuity member(s) of the heart valve prosthesis is achieved any excess individual strands or chordae may be divided and removed along with the tubes that pass through the body wall. This may be achieved by cutting the ends of the individual strands or chordae by opening the access tube under sterile conditions near to the skin and pushing the excess chordal material into the body prior to withdrawal of the access tubes in the fashion used for conventional chest drainage tubes.

### BRIEF DESCRIPTION OF THE DRAWINGS

Embodiments of the present invention will now be described by way of example, with reference to the accompanying drawings, in which:

FIG. 1 is a schematic illustration of the left side of a heart during diastole (relaxation of the left ventricle);

FIG. 2 is a schematic illustration of the left side of a heart during systole (contraction of the left ventricle);

FIG. 3 is a schematic illustration of a natural mitral valve seen from the left atrial aspect.

FIG. 4 is a schematic illustration of the natural anterior leaflet of a heart;

FIG. 5 is a schematic illustration of the natural posterior leaflet of a heart;

FIGS. 6, 7 and 8, are schematic illustrations of a valve according to an embodiment of the present invention;

FIG. 9 is a schematic illustration of the valve of FIG. 6, further illustrating an extra-cardiac restraining washer and crimping clip;

FIG. 10 is a schematic illustration of the valve of FIG. 6 shown attached to the papillary muscle of the heart;

FIGS. 11a-11d are schematic illustrations of a heart valve prosthesis in accordance with a preferred embodiment of the invention showing the prosthesis in closed position, and as constructed using a 3D spline curve, showing the prosthesis in the (a) x-y plane, (b) y-z plane, (c) x-z plane and (d) in rear view;

FIGS. 12a-12c show 2D plots for each plane describing the 3D spline curve defining the free edge of the valve elements of the valve of FIG. 11, wherein (a) x-y plane, (b) x-z plane, (c) y-z plane;

FIGS. 13a-13d are schematic illustrations of heart valve prostheses in accordance with embodiments of the present invention shown in closed position, and as constructed using a 3D spline curve and incorporating one or more folds into the free edge design of the valve, in particular (a) a basic 1-fold shape, (b) a 2-fold shape, (c) a 3-fold shape and (d) the 2-fold shape with additional cut-outs in the posterior leaflet;

FIGS. 14a and 14b are schematic illustrations of a heart valve prosthesis in accordance with an embodiment of the present invention, shown in closed position and depicting
(a) the inflow side of the closed valve, and
(b) the outflow side of the closed valve;

FIG. 15 is a schematic illustration of a heart valve prosthesis in accordance with an embodiment of the present invention, shown in open position;

FIGS. 16a and 16b show schematic illustrations of heart valve prostheses in accordance with embodiments of the present invention in both (a) closed and (b) open position;

FIGS. 17a and 17b are schematic illustrations of heart valve prostheses in accordance with embodiments of the present invention, shown in closed position and from outflow side;

FIGS. 18a and 18b are schematic illustrations of a heart valve prosthesis in accordance with embodiments of the present invention shown from outflow side in both (a) closed and (b) open position.

### DETAILED DESCRIPTION OF THE DRAWINGS

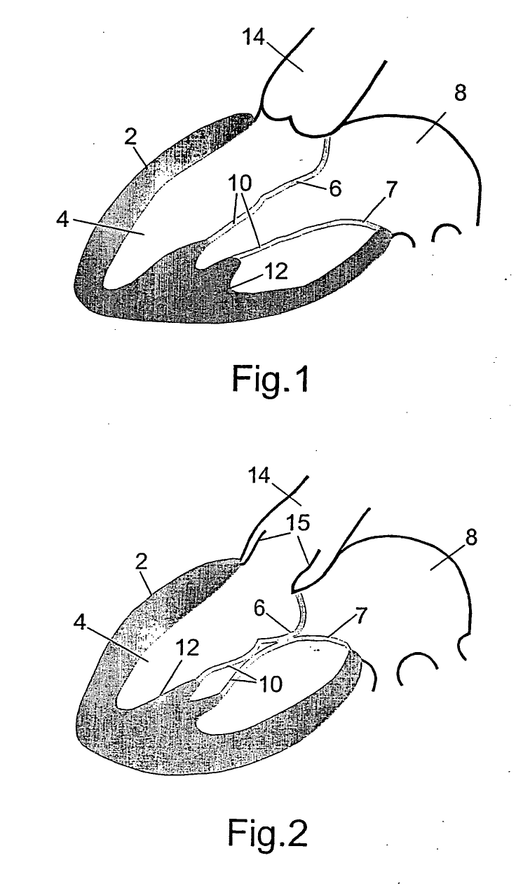

Referring first to FIG. 1, there is shown the left side of a healthy human heart 2 during diastole (relaxation of the left ventricle 4); the mitral valve leaflets 6, 7 are open to allow blood flow from the left atrium 8 to the left ventricle 4, and the chordae 10 attaching the leaflets 6 to the papillary muscles 12 are relaxed.

FIG. 2 depicts the left side of the heart 2 during systole (contraction of the left ventricle 4); the mitral valve leaflets 6, 7 are closed and the chordae 10 are tensed (preventing prolapse of the mitral leaflets 6, 7 into the left atrium 8 and ensuring that blood does not return to the left atrium 8 but is instead injected into the aorta 14).

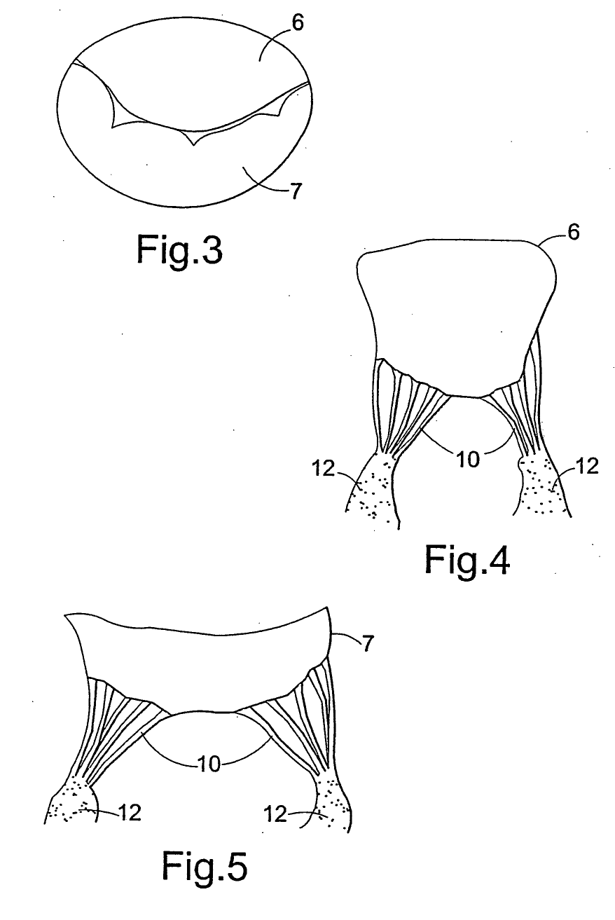

FIGS. 3, 4 and 5 depict the natural anterior and posterior leaflets 6, 7. The anterior leaflet 6 has less extensive attachment to the annulus than the posterior leaflet 7. Each leaflet has primary chordae tendineae 10, which attach to the papillary muscles 12.

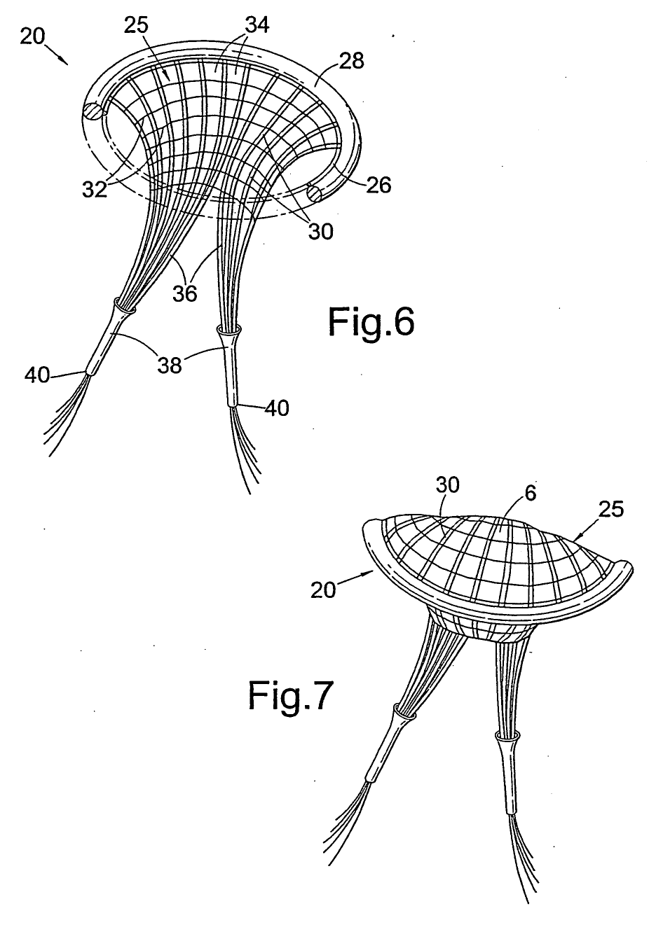

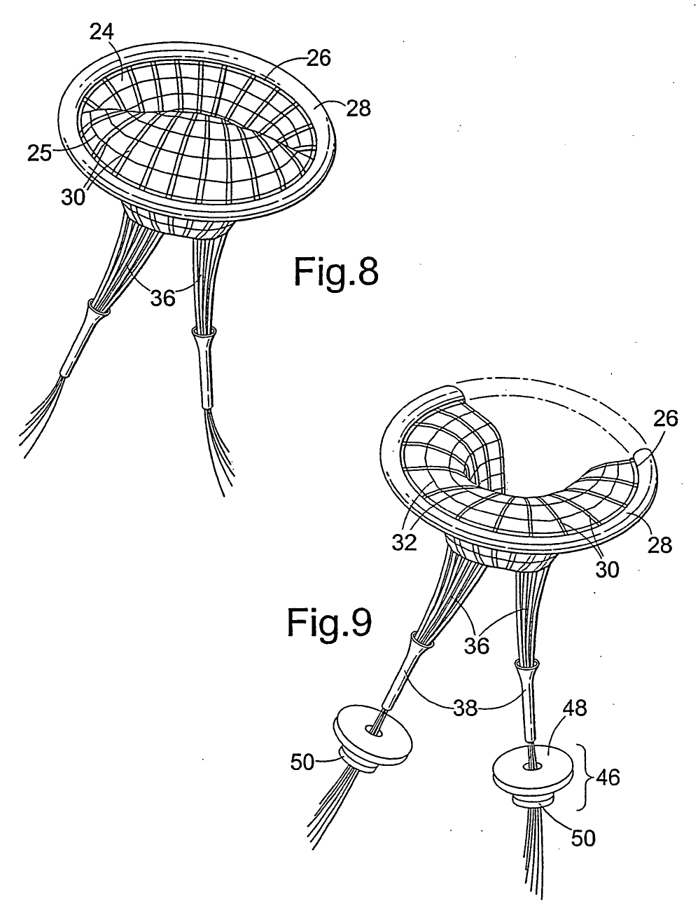

Reference is now made to FIGS. 6 and 7 of the drawings, which show views of a heart valve prosthesis 20 according to an embodiment of the present invention, and showing an anterior valve element 25, and also to FIG. 8 of the drawings, which also illustrates the posterior valve element 24.

The prosthesis 20 comprises a support in the form of a ring 26, which is incorporated into a fabric sewing ring 28. The sewing ring 28 shown in this embodiment is of polytetrafluoroethylene and facilitates the sewing of the heart valve prosthesis into the mitral orifice of the heart by a surgeon, as will be described. Each valve element 24, 25 features primary reinforcing strands 30, which extend from and are attached to the annular support ring 26. The strands 30 are of a strong flexible material, in this example polytetrafluoroethylene (PTFE). The primary strands 30 are linked by secondary strands 32, to create a net-like structure of a size and shape corresponding to the respective natural valve leaflet 6, 7. Each net-like structure is embedded within a thin layer 34 of a flexible biostable polymer, in this example polyurethane. The use of such a biostable polymer is advantageous in that no anticoagulant agent is required in use of the valve in vivo.

The reinforcing strands 30 continue beyond the valve elements 24, 25 and are grouped together to form two continuity members 36, which pass through respective tubes 38. As will be described, the members 36 are intended to serve a similar function to the primary chordae tendineae 10 of the natural valve. Each tube 38 has a leading end 40 of a form which facilitates the puncture of papillary muscle 12; the tube 38 is in the form of a trocar, which can be pushed through the heart muscle, ideally the thickest portion of the papillary muscle 12.

Reference is now also made to FIG. 9 of the drawings, which also depicts restraining means 46 for the continuity members 36, which means comprises a washer 48 and a compressible crimping clip 50 on each member 36. The restraining means 46 is intended to be located outside of the heart and to receive the continuity members 36 when the members 36 are passed through the tubes 38.

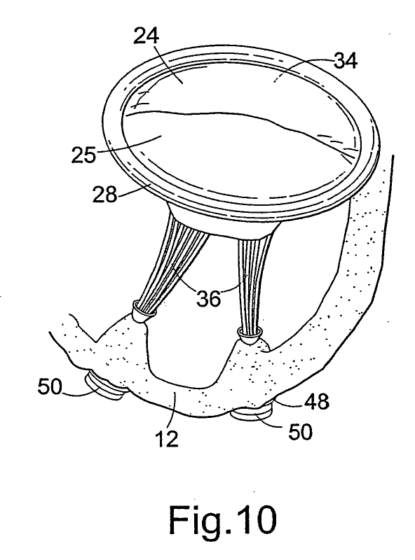

FIG. 10 depicts the heart valve prosthesis 20 secured to the papillary muscle 12 of a heart. It may be seen that the continuity members 36 are received by the tubes 38 which have been pushed through the papillary muscle 12.

In use, optimal geometry of the anterior and posterior valve elements 25 and 24, respectively, to mimic the function of the natural leaflets of the heart 6, 7, is achieved by adjustment of tension of the individual strands 30 of the continuity member 36 by the surgeon. The compressible crimping clips 50 restrain the strands-relative to the washers 48, compression of the clips 50 being undertaken by the surgeon once the strands 30 of the continuity members 36 have been tensioned appropriately.

Accurate tensioning of the continuity members 36 is achieved by making an initial adjustment on inspection of the valve elements 24, 25 with the heart still open, and fine tuned with the heart closed and beating normally, before closing the chest, using echocardiography to visualise the valve as it functions. Optimal leaflet geometry in the beating, working heart is achieved by selective tensioning of the individual strands or chordae of the continuity members 36, prior to crimping the clips 50.

The tension on the washers 48 prevents bleeding through and past the tubes 38, and the disposition of the fibre reinforcement of the anterior and posterior valve elements 25, 24, respectively allows even transmission of force from the distending valve elements as they are subjected to load by the contracting left ventricle 4. Furthermore, there is a near normal blood flow experienced across the prosthesis 20 and within the left ventricle, even in the mitral position, where flow is more complex involving vortices in the left ventricular cavity. Indeed, due to this more normal blood flow compared with conventional heart valve prostheses there is a decrease in thrombo-embolism and therefore a reduction in the thrombo-embolic risk associated with prosthetic valves. Importantly, the heart valve prothesis 44 maintains normal left ventricular-annular continuity, with preservation of left ventricular function. The adjustability of the heart prostheses, by selective tensioning of the continuity members 36, also allows the heart valve prosthesis 20 to be customised for the individual patient, for example, under echocardiographic control on the operating table.

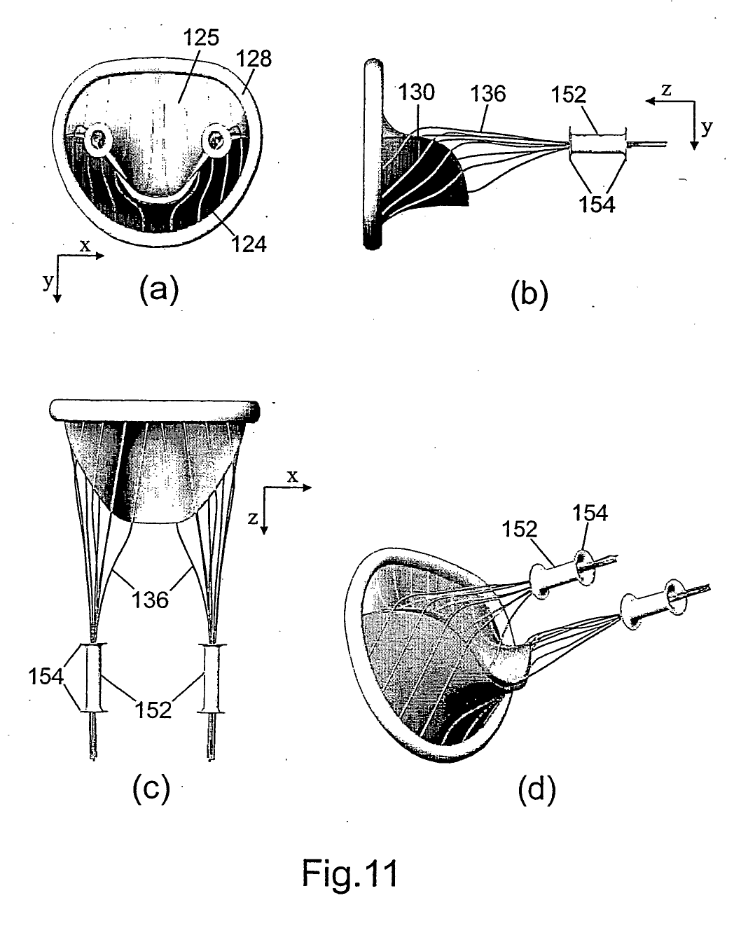

For clarity, the heart valve prostheses illustrated in FIGS. 11 to 18 show prostheses in the closed configuration with a small gap between the posterior and anterior free edges of the valve elements; in reality, in the closed configuration the free edges will be in contact.

FIGS. 11a-11d depict various views of a heart valve prosthesis in accordance with an embodiment of the present invention as designed using a 3D spline curve.

FIG. 11(a) depicts the x-y plane of the heart valve prosthesis comprising an anterior valve element ¹²⁵, posterior valve element 124, sewing ring 128 and primary reinforcing elements 130. The reinforcing elements 130 continue beyond the valve elements 124, 125 and are grouped together to form two continuity members 136, which pass through respective tubes 152. Each tube 152 comprises integral washers 154 for positioning the tube in relation to both the inside and outside of the ventricle of a patient's heart.

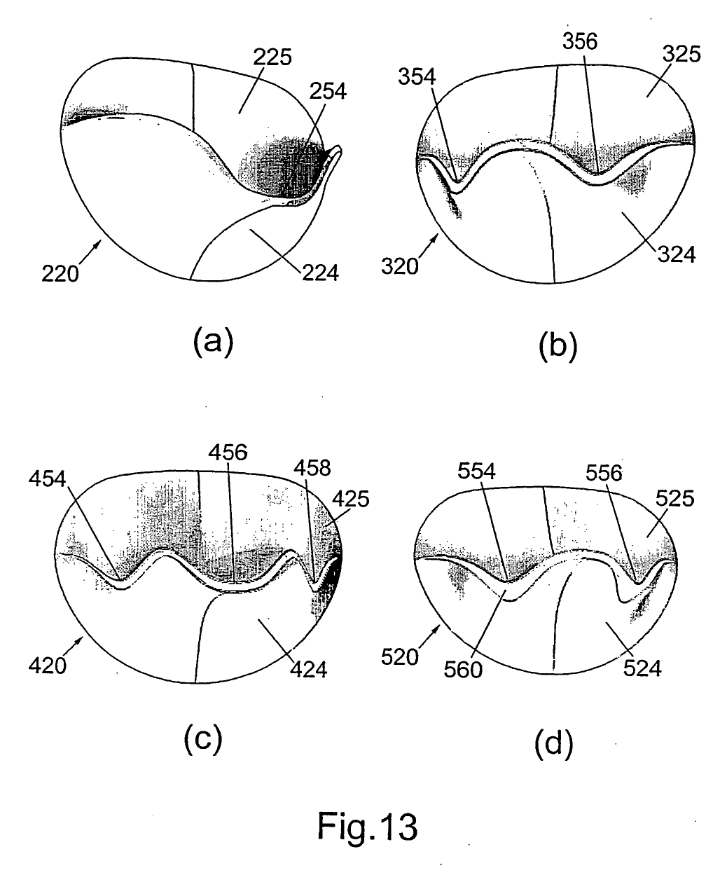

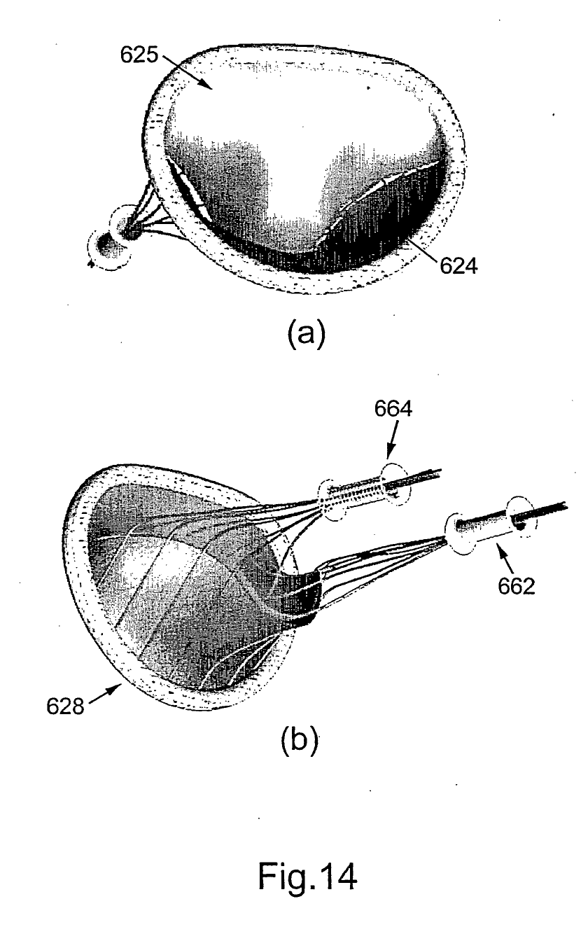

FIGS. 13a-13d depict various heart valve prostheses 220, 320, 420, 520 in accordance with embodiments of the invention comprising anterior and posterior valve elements 225, 224, 325, 324, 425, 424, 525, 524 respectively and showing one or more folds in the free edge. FIG. 13(a) depicts a basic shape of valve, which most closely mimics the natural valve, and comprising one fold 254, such that each valve element 224,225 defines a coapting surface having three points of inflection. FIG. 13(b) depicts a 2-fold design, with a two folds 354,356, and such that each valve element 324,325 has five points of inflection. FIG. 13(c) depicts a 3-fold design: a first fold 454, second fold 456 and third fold 458 are shown. FIG. 13(d) shows a 2-fold design, with a first fold 554, second fold 556 and cut-out section 560 in the posterior valve element 524.

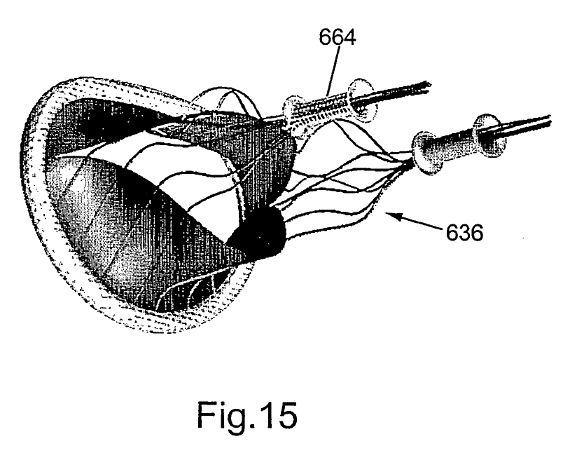

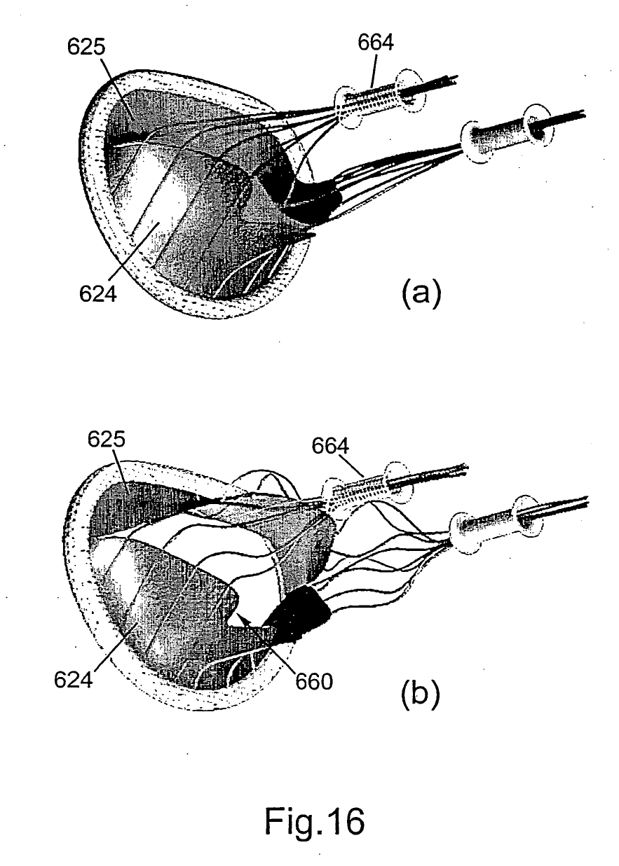

FIGS. 14-18 depict various views of heart valve prostheses in accordance with embodiments of the present invention with one or more folds in the free edge. FIGS. 14a and 14b depict a 1-fold design. FIG. 15 features a 2-fold design. FIGS. 16a and 16b, illustrate a prosthesis featuring a single fold, but with cut-outs in the posterior leaflets 624. It will be seen that in use, the introduction of cut-out sections in the posterior leaflet 624 of a 1-fold valve of FIG. 16b as compared with a 1-fold leaflet with no cut-outs in FIG. 14 provides a larger area of orifice. This allows a greater flow of blood through such an orifice without leakage of blood when the valve is closed.

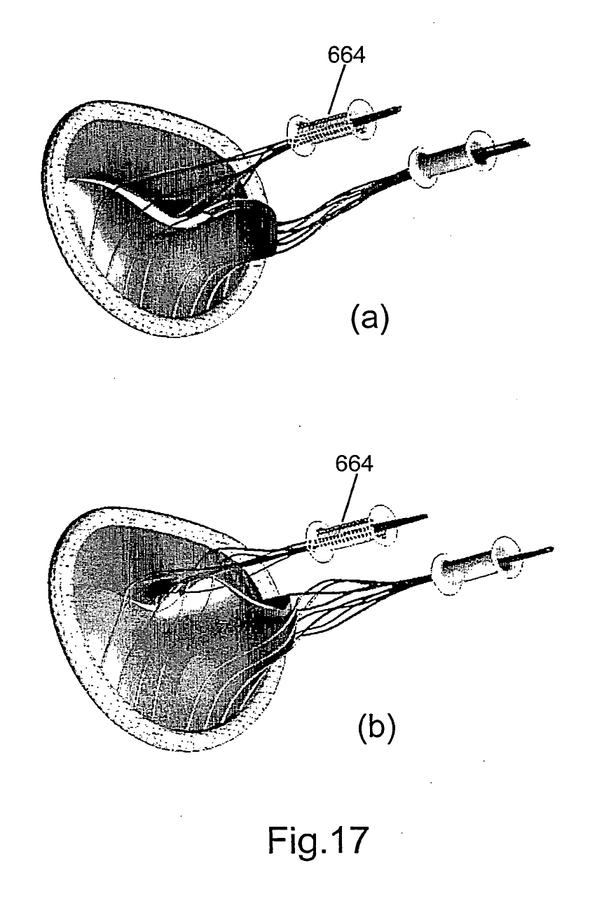

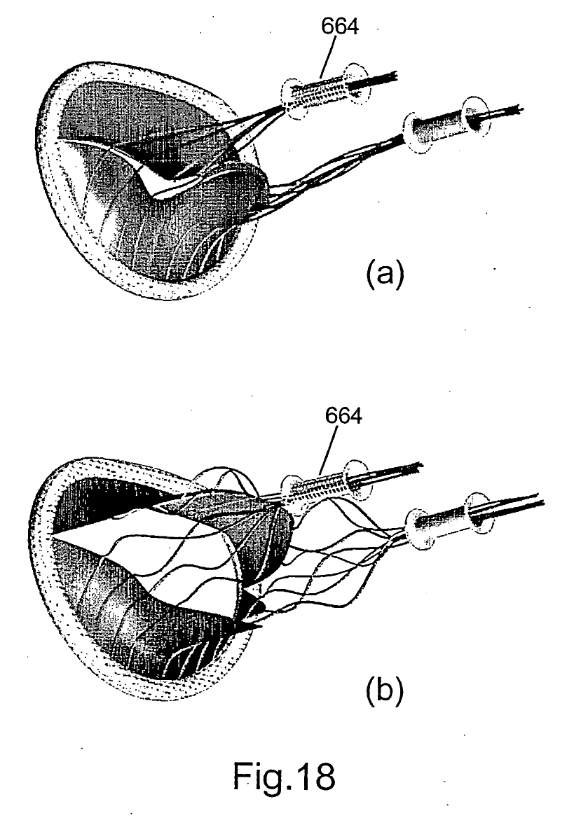

FIGS. 17a and 17b show heart valve prostheses of 2-fold and 3-fold shape, respectively. FIGS. 18a and 18b show a heart valve prosthesis of 2-fold shape with cut-outs in the posterior leaflet. These embodiments also feature tubular members 664 having a mesh-like structure, which may encourage tissue ingrowth.

Various modifications may be made to the embodiments hereinbefore described without departing from the scope of the present invention.

### EXAMPLES

Mathematical Representations of Valve Designs in Closed Position.

The mathematically modelled mitral valve designs are based on the physical model of a mitral valve prosthesis in accordance with an embodiment of the invention moulded out of Woods metal. The designs are all symmetrical about a centre line, so it is sufficient to construct one half of the posterior and anterior leaflets, and then mirror these to complete the designs. For example, the posterior half-leaflet (of FIG. 11) is constructed as follows. The portion of the annulus perimeter curve is drawn first, from the centre-line position to the commisure where it meets the anterior region. The centre-line curve then is drawn at right angles from the annulus curve centre point. Finally, a 3D curve is drawn to connect the centre-line curve to the commissure. This 3D curve is a half-section of the free edge of the leaflet and is constructed by means of three 2D boundary curves Using these three boundary curves, it is possible to create a 3D surface representing the leaflet half-section. This surface is mirrored to complete the leaflet. The anterior half-section is constructed in a similar fashion. For greater local control of curvature along the leaflet, additional guide or constraint curves are used.

1. The Free Edge

The free edge of the mitral valve design is constructed using a 3D spline curve. Because the spline curve travels through 3D space, it is difficult to obtain a single equation to define it. Polynomial equations of the 6^{th} order representing the free edge curve as it is projected on to the standard Cartesian planes have been defined, that is, the x-y plane, x-z plane, and y-z plane.

These three 2D equations describe the 3D spline curve defining the free edge. The equations represent one-half of the leaflet free edge, which is symmetrical about the centre plane, that is, the y-z plane.

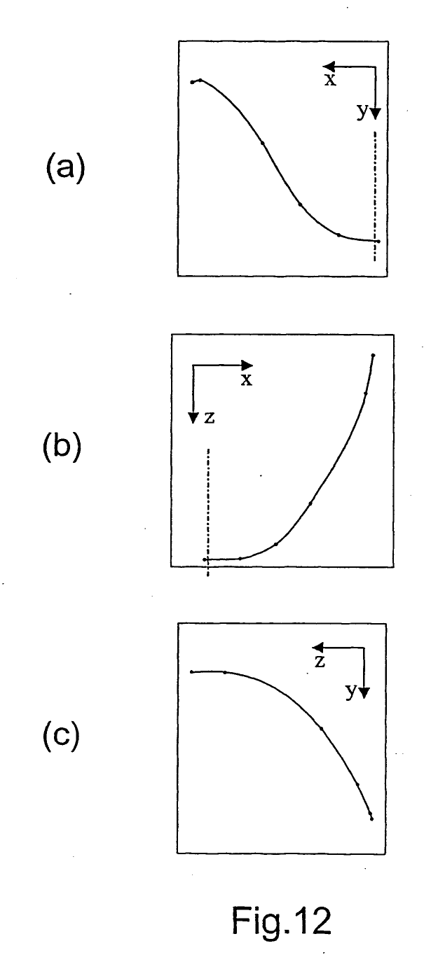

The (x, y, z) coordinates of the points on the 3D spline have been tabulated. 2D plots for each plane accurately represent the curves seen in FIG. 12. Sixth order polynomial trendlines were graphed and equations for these trendlines were verified by re-graphing the curves and comparing with the original model. Typical curves for the 1-fold leaflet design are demonstrated in FIG. 12 for the three planes. The coefficients may be adjusted to optimise the fit of the trendline curve to the original model.

The general 6^{th} order equation that represents the free edge for any embodiment is:
f(t)=at⁶+bt⁵+ct⁴+dt³+et²+ft+g
where t is the x-, y-, or z-coordinate of a point along the free edge curve, f(t) is a function of this point, and a-g are coefficients.

The sixth order equation allows for a range of design variants, with the possibility of incorporating one or more folds into the leaflet free edge design (seen clearly from the x-y plane, looking directly at valve from the outflow side), cut-outs (seen clearly from the x-z plane), or any combination of these. The curves may be designed by suitable modifications of the coefficients of the equation. For example, as seen in FIG. 13, four examples of such embodiments: (a) a basic 1-fold shape; (b) a 2-fold shape; (c) a 3-fold shape and (d) the 2-fold shape with additional cut-outs in the posterior leaflet.

For clarity, all of the designs as depicted in FIG. 13 show a small gap between the posterior and anterior free edges. In reality, the designs represent the closed configuration of the valve and the free edges will be in contact and, thus, the representative polynomial equations will be the same. In the case where, for example, the posterior leaflet incorporates a number of cut-outs and the anterior leaflet does not, the resulting equations will be different, with the anterior leaflet retaining the non-cut-out equation. Cut-outs may be applied to one or both leaflets.

X-y Plane

By modifying the coefficients of the general equation, many complex designs are possible. The following table demonstrates values of the coefficients that represent 1-, 2-, and 3-fold leaflet designs:

| | Design | | |
|---|---|---|---|
| Coefficient | 1-fold leaflet | 2-fold leaflet | 3-fold leaflet |
| a | 0.009 × 10⁻⁵ | −1.54 × 10⁻⁵ | 1.93 × 10⁻⁵ |
| b | −3.35 × 10⁻⁵ | 91.8 × 10⁻⁵ | −90 × 10⁻⁵ |
| c | 0.0017 | −0.0195 | 0.0133 |
| d | −0.0278 | 0.1721 | −0.054 |
| e | 0.1 | −0.57 | −0.15 |
| f | −0.1991 | 0.7 | 0.37 |
| g | 24.0 | 15.8 | 15.8 |

The value of the coefficient g represents the y-coordinate at the centre-line, that is, it defines the offset distance in the y-direction from the origin, at the lowest point of the anterior annulus. Free edge cut-outs cannot be clearly seen from this plane. The best plane of view for cut-outs is the x-z plane.
x-z Plane

The following table shows the values of the coefficients defining the free edge equation from the x-z plane, with and without a cut-out:

Design

| Coefficient | Without Cut-out | With Cut-out |
|---|---|---|
| a | −1.0 × 10⁻⁵ | 1.93 × 10⁻⁵ |
| b | 50 × 10⁻⁵ | −90 × 10⁻⁵ |
| c | −0.009 | 0.133 |
| d | 0.0693 | −0.053 |
| e | −0.265 | −.02 |
| f | 0.321 | 0.15 |
| g | 20.0 | 20.0 |

Viewed from this plane, a design incorporating multiple folds appears similar to a design with a single fold. Again, the g coefficient represents an offset from the origin, in this case the offset distance in the z-direction along the centre-line. This equates to the maximum depth of the free edge from the annulus.
y-z Plane

The following table demonstrates values of co-efficients that represent the free edge equation for embodiments of 1-2- and 3-fold leaflet designs:

Design

| Coefficient | 1-fold leaflet | 2-fold leaflet | 3-fold leaflet |
|---|---|---|---|
| a | 0.3 × 10⁻⁵ | 0.22 × 10⁻⁵ | −0.01 × 10⁻⁵ |
| b | −18.6 × 10⁻⁵ | −7 × 10⁻⁵ | 40 × 10⁻⁵ |
| c | 0.0043 | −8 × 10⁻⁵ | −0.0007 |
| d | −0.045 | 0.0155 | 0.0111 |
| e | 0.245 | −0.07 | 0.238 |
| f | −0.5 | 0.25 | −0.56 |
| g | 11.0 | 11.0 | 11.0 |

The g coefficient represents the y-coordinate where the curve begins at the annulus, that is, the commissure position for the leaflets.

It may be the case that one or more of the coefficients (a, b, etc.) for free edge curves have a zero value, which would cause the associated terms of the 6 order equation to tend to zero.

2. Centre-Line Curves

The centre-line curve extends from the annulus in the z-direction to intersect with the start of the free edge 3D curve. This curve is significantly different for posterior and anterior leaflets, but both are in the y-z plane only and can be defined by a single equation representing a 2D curve.

Again, the curve can be expressed as a 6^{th}-order polynomial equation of the form:
y=az⁶+bz⁵+cz⁴+dz³+ez²+fz+g

However, the curve is generally less complex and, for most embodiments, the higher terms have coefficients that tend towards zero. The following table demonstrates coefficients of the posterior and anterior centre curve equations suitable for 1-, 2- and 3-fold leaflet designs:

| | Design | | | |
|---|---|---|---|---|
| | Posterior Leaflet | | Anterior Leaflet | |
| Coefficient | 1-fold | 2- and 3-fold | 1-fold | 2- and 3-fold |
| a | 0.0 | 0.0 | 0.0 | 0.0 |
| b | 0.0 | 0.0 | 0.0 | 0.0 |
| c | 0.0003 | 0.0005 | −0.0012 | −0.0009 |
| d | −0.015 | −0.026 | 0.058 | 0.042 |
| e | 0.026 | 0.49 | −1.02 | −0.69 |
| f | −1.8 | −4.0 | 7.5 | 4.94 |
| g | 28.0 | 28.0 | 0.0 | 0.0 |

As with previous polynomial equations, the value of the g coefficient represents the starting y coordinate at the intersection with the annulus. Therefore, the start position of the posterior curve is at the greatest height on the annulus, that is, 28. The anterior curve starts at the origin.

3. Constraint Curves

As mentioned earlier, three boundary conditions are sufficient to create the surface of the leaflet, that is, the free edge curve, the centre-line curve, and the annulus boundary. Greater control of the region within this surface can be achieved by introducing a number of guide, or constraint, curves. These can be of any number or orientation, from 2D to 3D and in any direction, but for practical use, they are generally parallel to the centre-line curve and offset towards the commissural edge. They will be similar to the centre-line curve, but will have small changes in the coefficients to account for the starting y coordinate at the annulus and end position at the free edge.

Mitral Valve Prosthesis

Method of Insertion:

The diseased valve is excised, including the anterior and posterior leaflets and chordae, leaving the tips of the papillary muscles and a rim of leaflet tissue at the mitral orifice intact. A valve sizer, consisting of a ring of similar shape to that of the ring of the prosthesis, together with two prongs which act as guides to the location of the new chordal insertion sites, is used to judge the appropriate size, orientation and location of chordal insertion sites for the new prosthesis. A long needle is used to perforate the heart at the intended chordal insertion sites, as near to the papillary muscle tips as possible, allowing a flexible guide-wire to be passed from inside the heart to the outside. This is used to guide the passage of dilators, as may be necessary, and finally the two tubular inserts through which the chordae of the new valve are passed before the sewing ring is secured.

The new valve is secured in the appropriate orientation (mimicking the natural valve orientation) by suturing the sewing ring of the new valve prosthesis to the mitral annulus of the heart in the standard fashion used for other artificial heart valves, according to surgical preference and practice.

The two groups of artificial chordae, which have been passed through the tubular inserts to the outside of the heart, are then passed through tubes, which may traverse the body wall (similar to the chest drainage tubes which are in routine use). This allows the chordae to be tensioned approximately from outside the operative field to make the leaflets of the valve prosthesis close evenly, in the plane of the valve annulus, without being too loose to cause prolapse of the leaflets into the atrium, or too tight to interfere with leaflet apposition, as judged by the surgeon. The chordae are then temporarily clamped outside the heart, to maintain tension until final adjustments are made. The operation is completed in the standard fashion.

Methods of Adjusting the Valve:

The valve may be adjusted after its insertion by using echocardiographic imaging. This adjustment may be made in the operating room before closure of the chest, with the aid of (for example) transoesophageal echocardiographic imaging, or in the first few postoperative days using echocardiographic imaging (or other forms of imaging) by tensioning or slackening the synthetic chordae. The chordae, which are passed to the outside of the body through the tubes inserted by the surgeon, are tensioned or slackened, individually or in groups, as required to achieve optimal leaflet apposition as defined by echocardiography or other imaging method. Once the best degree of tensioning has been achieved, the chordae are secured at the surface of the heart by a suitable device that is actuated from outside the body, and the excess chordae outside the heart are divided and removed along with the tubes that pass through the body wall.

1. A heart valve prosthesis comprising:
a support adapted to be coupled to the mitral annulus;
a valve element coupled to the support and moveable between an open position and a closed position;
at least one continuity member having one end coupled to said support and the other end for coupling to papillary muscle; and
restraining means for securing the continuity member to an area of heart muscle and for permiitting adjustment of the continuity member externally of the heart.

2. A heart valve prosthesis comprising:
a support adapted to be coupled to the mitral annulus; first and second valve elements coupled to the support
and moveable between an open position and a closed position, the elements defining curved coapting surfaces, each surface having at least two points of inflection; and
at least one continuity member having one end coupled to said support and the other end adapted for coupling to papillary muscle.

3. The prosthesis of claim 1, wherein the support has a ring-like structure.

4. The prosthesis of claim 1 wherein the support comprises a semi-rigid structure.

5. The prosthesis of claim 1 wherein the support is incorporated into a fabric sewing ring.

6. The prosthesis of claim 5, wherein said fabric sewing ring is formed of polytetrafluoroethylene.

7. The prosthesis of claim 1, wherein the valve element is flexible.

8. The prosthesis of claim 1, wherein a plurality of valve elements are provided.

9. The prosthesis of claim 1 wherein two flexible valve elements are provided.

10. The prosthesis of claim 9, wherein the valve elements are formed and arranged to mimic the function of the anterior and posterior leaflets of a working biological valve in vivo.

11. The prosthesis of claim 1, wherein the valve element comprises elongate reinforcing elements.

12. The prosthesis of claim 11, wherein the reinforcing elements are in the form of elongate strands.

13. The prosthesis of claim 12, wherein the strands are multifilament.

14. The prosthesis of claim 11, wherein the reinforcing elements are of at least one of polytetrafluoroethylene (PTFE), polypropylene and carbon fibre.

15. The prosthesis of claim 11, wherein the reinforcing elements are attached around the support and radiate therefrom.

16. The prosthesis of claim 11 wherein the reinforcing strands are embedded in a single plane within the valve element.

17. The prosthesis of any of claim 11 wherein the reinforcing strands lie in at least two separate planes with polymer material therebetween.

18. The prosthesis of claim 15, wherein the reinforcing elements comprise primary elements extending in one direction and secondary elements extending in another direction.

19. The prosthesis of claim 11, wherein the reinforcing elements comprise short strand discontinuous fibres of high aspect ratio.

20. The prosthesis of claim 19, wherein the short strand discontinuous fibres of high aspect ratio are nanotubes.

21. The prosthesis of claim 18, wherein primary elements are mutually coupled by secondary elements to create a netlike structure.

22. The prosthesis of claim 21, wherein the net-like structure is coupled to a flexible material forming at least one surface of the valve element.

23. The prosthesis of claim 21, wherein the net-like structure is embedded within a biostable polymer.

24. The prosthesis of claim 23, wherein the polymer comprises polyurethane.

25. The prosthesis of claim 21, wherein the net-like structure is of a size and shape corresponding to the valve element.

26. The prosthesis of claim 11, wherein the reinforcing elements continue beyond the valve element and ends of the elements form continuity members.

27. The prosthesis of claim 1, further including restraining means comprising a tubular member for receiving the other end portion of the continuity member.

28. The prosthesis of claim 27, wherein the tubular member is a tube which is at least semi-rigid.

29. The prosthesis of claim 27 wherein the tubular member is of a metal alloy.

30. The prosthesis of claim 29 wherein the alloy is Titanium.

31. The prosthesis of claim 29 wherein the alloy is Nitinol.

32. The prosthesis of claim 27, wherein the tubular member has an open mesh structure.

33. The prosthesis of claim 32, wherein the open mesh structure is adapted to permit tissue ingrowth.

34. The prosthesis of any of claims 27 wherein the tubular member comprises at least one integral flange.

35. The prosthesis of claim 34 wherein the integral flange comprises a mesh structure.

36. The prosthesis of claim 1 wherein the prosthesis is coated with a material selected to improve blood compatibility.

37. The prosthesis of claim 36 wherein the material to improve blood compatibility is one of a hydrogel and a fluoropolymer plasma.

38. The prosthesis of claim 28, wherein the leading end of the tube is in the form of a trocar, adapted to be pushed through the papillary muscle.

39. The prosthesis of claim 1, further comprising restraining means for securing the continuity member to an area of heart muscle and for permitting adjustment of the continuity member externally of the heart.

40. The prosthesis of claim 39, wherein the restraining means comprises a compressible crimping clip.

41. The prosthesis of claim 39 or 40, wherein the restraining means comprises a flange.

42. The prosthesis of claim 1 comprising at least first and second valve elements having free ends defining at least one fold.

43. The prosthesis of claim 42 wherein the valve elements comprise cut-out regions at free edges of at least one valve element.

44. The prosthesis of claim 43 wherein cut-out regions are provided in alternate folds in both valve elements.

45. The prosthesis of claim 1 wherein a free edge of the valve element is constructed using a 3D spline curve.

46. The prosthesis of claim 45 wherein the free edge curve with at least one fold is defined as:
f(t)=at⁶+bt⁵+ct⁴+dt³+et²+ft+g
where t is the x-, y-, or z-coordinate of a point along the free edge curve, f (t) is a function of this point, and a-g are coefficients.

47. A method of inserting a heart valve prosthesis, the prosthesis comprising a support, a valve element coupled to the support and moveable between an open position and a closed position, and at least one continuity member having one end coupled to said support, said method comprising the steps of:
(i) securing the support to the mitral orifice of the heart;
(ii) passing the other end of the continuity member through the papillary muscle of the heart; and
(iii) adjusting the continuity member externally of the heart.

48. A method of inserting a heart valve prosthesis, the prosthesis comprising a support, a valve element coupled to the support and moveable between an open position and a closed position, and at least one continuity member having one end coupled to said support, said method comprising the steps of:
(a) excising a diseased valve from a patient, wherein papillary muscle tips and a rim of leaflet tissue at the mitral orifice are left intact;
(b) measuring the size of the patient's valve cavity;
(c) determining appropriate sites within the patient's papillary muscle to receive the continuity member;
(d) perforating the patient's heart at the determined papillary muscle site;
(e) securing the support to the mitral orifice of the heart;
(f) passing the continuity member through the papillary muscle of the heart;
(g) adjusting the continuity member externally of the heart; and
(h) securing the continuity member to said papillary muscle.

49. The method of claim 47, further comprising the steps of: closing the patient's chest; and then further adjusting the continuity member.

50. The method of claim 47, further comprising the step of:
individually adjusting strands of the continuity member.
